(19) United States
(12) Patent Application Publication
(10) Pub. No.: US 2024/0335432 A1
(43) Pub. Date: Oct. 10, 2024

(54) THERAPEUTIC COMPOUND FOR NEURONAL CEROID LIPOFUSCINOSIS

(71) Applicants: JCR PHARMACEUTICALS CO., LTD., Ashiya-shi (JP); ARMAGEN, INC., San Diego, CA (US)

(72) Inventor: Mathias SCHMIDT, San Diego, CA (US)

(73) Assignees: JCR PHARMACEUTICALS CO., LTD., Ashiya-shi (JP); ARMAGEN, INC., San Diego, CA (US)

(21) Appl. No.: 18/294,079

(22) PCT Filed: Sep. 2, 2022
(86) PCT No.: PCT/US2022/042425

§ 371 Date: Jan. 31, 2024

Related U.S. Application Data
(63) Continuation of application No. 17/465,478, filed on Sep. 2, 2021, now abandoned.

(51) Int. Cl. A61K 31/4375; A61P 25/28

(52) U.S. Cl. CPC A61K 31/4375; A61P 25/28

(57) ABSTRACT

[Problem to be solved] To provide a therapeutic compound for neuronal ceroid lipofuscinosis. [Solution] Provided is a compound for treatment and/or prevention of neuronal ceroid lipofuscinosis represented by the following general formula [I]. In the general formula [I]. R1 is a methyl group or a hydroxymethyl group, R2 is a methyl group or a hydroxymethyl group, R3 is one of a hydroxy group, a methoxy group, an ethoxy group, an isopropoxy group, a methoxyethoxy group, a methoxypropoxy group, or an ethoxypolopoxy group, and R4 is one of a hydroxy group. a methoxy group, an ethoxy group, an isopropoxy group, a methoxyethoxy group, a methoxypropoxy group, or an ethoxypolopoxy group, In particular, provided is a compound represented by the general formula [I] with R1 being a methyl group, R2 being a methyl group, R3 being a hydroxy group, and R4 being a methoxyethoxy group.

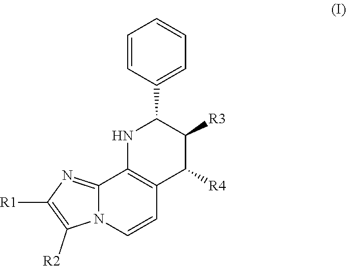

### TECHNICAL FIELD

The present invention relates to a compound for treatment of neuronal ceroid lipofuscinosis.

### TECHNICAL BACKGROUND

Neuronal ceroid lipofuscinosis (NCL) or Batten disease, which is a type of lysosomal disease, is a hereditary disease characterized by progressive neurodegeneration in which, due to mutations in any of multiple causative genes such as CLN1, CLN2, and CLN3, lipofuscin accumulates in intracellular lysosomes, causing blindness, muscle coordination ataxia, mental retardation or decreased mental function, emotional disturbance, seizures, muscle spasms, decreased muscle tone, movement disorders, and the like. It is classified into 14 types according to genes that show abnormalities.

Neuronal ceroid lipofuscinosis includes an infant type (Santavuori-Haltia disease), a late infant type (Jansky-Bielshowsky disease), a juvenile type (Spielmeyer-Vogt disease), and an adult type (Kufus disease or Parry disease).

Enzyme replacement therapy using recombinant TPP1 (Cerliponase alfa, BioMarin Pharmaceutical) for treatment of neuronal ceroid lipofuscinosis type 2 has recently been approved in the United States, Europe, and Japan and administration using an intracerebroventricular administration system has been performed (Patent Document 1). However, for other types of neuronal ceroid lipofuscinosis, currently, there is no effective treatment or only symptomatic treatment or supportive treatment is available.

Soraprazan is a gastric H+/K+ ATPase inhibitor and acts as an inhibitor of gastric acid secretion (Non Patent Document 1).

### RELATED ART

### Patent Document

[Patent Document 1] International Publication No. 2016/182862.

### Non Patent Document

[Non Patent Document 1] Simon WA. J Pharmacol. Exp Ther. 321. 866-74 (2007)

### SUMMARY OF THE INVENTION

### Problems to be Solved by the Invention

An objective of the present invention is to provide a therapeutic compound for neuronal ceroid lipofuscinosis.

### Means for Solving the Problems

In the research aimed at the above object, the present inventors have found that a compound according to the present invention can reduce lipofuscin accumulated in cells of a patient with neuronal ceroid lipofuscinosis, and thus accomplished the present invention. That is, the present invention includes the following.
1. A compound for treatment and/or prevention of neuronal ceroid lipofuscinosis represented by the following general formula [I] wherein in the general formula [I], R1 is a methyl group or a hydroxymethyl group, R2 is a methyl group or a hydroxymethyl group, R3 is one of a hydroxy group, a methoxy group, an ethoxy group, an isopropoxy group, a methoxyethoxy group, a methoxypropoxy group, or an ethoxypolopoxy group, and R4 is one of a hydroxy group, a methoxy group, an ethoxy group, an isopropoxy group, a methoxyethoxy group, a methoxypropoxy group, or an ethoxypolopoxy group.

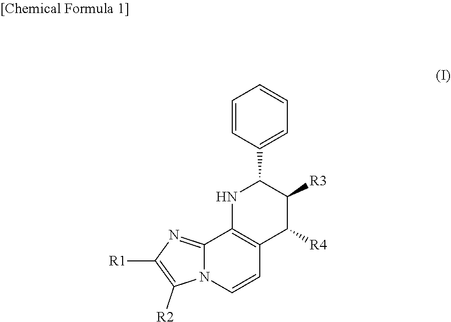

2. The compound according to 1 above, wherein, in the general formula [I], R1 is a methyl group.
3. The compound according to 1 above, wherein, in the general formula [I], R1 is a methyl group, and R2 is a methyl group.
4. The compound according to 1 above, wherein, in the general formula [I], R3 is a hydroxy group.
5. The compound according to 1 above, wherein, in the general formula [I], R3 is a hydroxy group, and R4 is a methoxyethoxy group.
6. The compound according to 1 above, wherein, in the general formula [I], R1 is a methyl group, R2 is a methyl group, R3 is a hydroxy group, and R4 is a methoxyethoxy group.
7. A pharmaceutical composition for treatment and/or prevention of neuronal ceroid lipofuscinosis, comprising a therapeutically effective amount of the compound according to any one of 1 to 6 above or a pharmaceutically acceptable salt thereof.
8. The pharmaceutical composition according to 7 above to be administered to a patient with neuronal ceroid lipofuscinosis for reducing the amount of lipofuscin accumulated in the patient's body.
9. The pharmaceutical composition according to 8 above, wherein the administration is subcutaneous, intravenous, intramuscular, parenteral, local, oral, transdermal, intraperitoneal, intraorbital, implantation, inhalation, intraarachnoid, intravesical, or intranasal administration.
10. The pharmaceutical composition according to 7 above, being in a dosage form selected from a group consisting of a tablet, a capsule, a granule, powder, an oral solution, a syrup, an oral jelly, an oral tablet, an oral spray, an oral semi-solid preparation, an orally disintegrating tablet, a parenteral emulsion, a parenteral suspension, a parenteral solution, an effervescent tablet, a mouthwash, an injection, and an inhalant.
11. The pharmaceutical composition according to any one of 7 to 10 above, further comprising a pharmaceutically acceptable additive.
12. The pharmaceutical composition according to 11 above, wherein the additive is selected from a group consisting of a flavoring agent, a foaming agent, a fragrance, an excipient, an isotonic agent, a surfactant, an emulsifier, a colorant, a buffer, zinc or its salt, a preservative, an antioxidant, a pH adjusting agent, a stabilizer, a suspension agent, a solubilizing agent, and a mixture at least two of aforementioned additives.
13. The pharmacological composition according to any one of 7 to 12 above, being used together with enzyme replacement therapy for a patient with neuronal ceroid lipofuscinosis.

### Effect of the Invention

According to the present invention, a novel therapeutic agent for neuronal ceroid lipofuscinosis can be provided.

### BRIEF DESCRIPTION OF DRAWINGS

FIG. 1 shows images of the autofluorescence observation of NHDF cells obtained in Example 2-4. From left to right, the images are the autofluorescent images of NHDF cells cultured in the media containing soraprazan at the concentrations of 0 (Untreated), 0.1, 1, and 10 μmol/L, respectively. The images are shown in pairs except the cells cultured in the presence of 1 μmol/L soraprazan.

FIG. 2 shows images of the autofluorescence observation of CLN2 cells (GM09404) obtained in Example 2-4. From left to right, the images are the autofluorescent images of CLN2 cells (GM09404) cultured in the media containing soraprazan at the concentrations of 0 (Untreated), 0.1, 1, and 10 μmol/L, respectively. The images are shown in pair.

FIG. 3 shows images of the autofluorescence observation of CLN2 cell (GM09668) obtained in Example 2-4. From left to right, the images are the autofluorescent images of CLN2 cells (GM09668) cultured in the media containing soraprazan at the concentrations of 0 (Untreated), 0.1, 1, and 10 μmol/L, respectively. The images are shown in pair.

FIG. 4 shows images of the autofluorescence observation of CLN2 cell (GM09669) obtained in Example 2-4. From left to right, the images are the autofluorescent images of CLN2 cells (GM09669) cultured in the media containing soraprazan at the concentrations of 0 (Untreated), 0.1, 1, and 10 μmol/L, respectively. The images are shown in pair.

FIG. 5 shows images of the autofluorescence observation of CLN2 cell (GM10570) obtained in Example 2-4. From left to right, the images are the autofluorescent images of CLN2 cells (GM10570) cultured in the media containing soraprazan at the concentrations of 0 (Untreated), 0.1, 1, and 10 μmol/L, respectively. The images are shown in pair.

FIG. 6 shows images of the autofluorescence observation of CLN2 cell (GM10672) obtained in Example 2-4. From left to right, the images are the autofluorescent images of CLN2 cells (GM10672) cultured in the media containing soraprazan at the concentrations of 0 (Untreated), 0.1, 1, and 10 μmol/L, respectively. The images are shown in pair.

FIG. 7 shows images of the autofluorescence observation of CLN2 cell (GM16485) obtained in Example 2-4. From left to right, the images are the autofluorescent images of CLN2 cells (GM16485) cultured in the media containing soraprazan at the concentrations of 0 (Untreated), 0.1, 1, and 10 μmol/L, respectively. The images are shown in pair.

FIG. 8 shows images of the autofluorescence observation of CLN2 cell (GM16486) obtained in Example 2-4. From left to right, the images are the autofluorescent images of CLN2 cells (GM16486) cultured in the media containing soraprazan at the concentrations of 0 (Untreated), 0.1, 1, and 10 μmol/L, respectively. The images are shown in pair.

FIG. 9 shows images of the autofluorescence observation of CLN2 cell (GM09668) obtained in Example 2-6. From left to right, the images are the autofluorescent images of CLN2 cells (GM09668) cultured in the media containing soraprazan at the concentrations of 0 (Untreated), 0.1, 1, and 10 μmol/L, respectively. The images are shown in pair.

FIG. 10 shows images of the autofluorescence observation of CLN2 cell (GM09669) obtained in Example 2-6. From left to right, the images are the autofluorescent images of CLN2 cells (GM09669) cultured in the media containing soraprazan at the concentrations of 0 (Untreated), 0.1, 1, and 10 μmol/L, respectively. The images are shown in pair.

FIG. 11 shows images of the autofluorescence observation of CLN2 cell (GM10570) obtained in Example 2-6. From left to right, the images are the autofluorescent images of CLN2 cells (GM10570) cultured in the media containing soraprazan at the concentrations of 0 (Untreated), 0.1, 1, and 10 μmol/L, respectively. The images are shown in pair.

FIG. 12 is a bar graph showing the ratio of the area where fluorescent was detected to the total area of the image (% Fluorescent Area) calculated from the images of FIGS. 9-11. The vertical axis indicates % Fluorescent. Black bars indicate % Fluorescent Area of the cells cultured in the presence of 0 μmol/L soraprazan, hatched bars indicate % Fluorescent Area of the cells cultured in the presence of 1 μmol/L soraprazan, and white bars indicate % Fluorescent Area of the cells cultured in the presence of 10 μmol/L soraprazan. Average values are shown with standard errors (vertical lines).

### MODE FOR CARRYING OUT THE INVENTION

A compound according to the present invention is able to be represented by the following general formula [I].

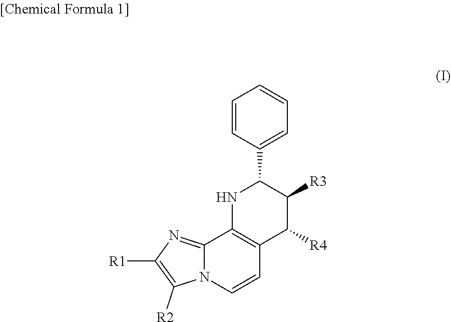

In the above general formula [I], R1 is not particularly limited, but is preferably a methyl group or a hydroxymethyl group, and more preferably a methyl group.

In the above general formula [I], R2 is not particularly limited, but is preferably a methyl group or a hydroxymethyl group, and more preferably a methyl group.

In the above general formula [I], R3 is not particularly limited, but is preferably a hydroxy group, a methoxy group, an ethoxy group, an isopropoxy group, a methoxyethoxy group or a methoxypropoxy group, more preferably a hydroxy group, a methoxy group or an ethoxy group, and even more preferably a hydroxy group.

In the above general formula [I], R4 is not particularly limited, but is preferably a hydroxy group, a methoxy group, an ethoxy group, an isopropoxy group, a methoxyethoxy group or a methoxypropoxy group, more preferably a methoxyethoxy group or a methoxypropoxy group, and even more preferably a methoxyethoxy group.

A compound represented by a chemical formula [II], corresponding to the above general formula [I] wherein R1 is a methyl group, R2 is a methyl group, R3 is a hydroxy group, and R4 is a methoxyethoxy group, is a compound known by the name soraprazan or BYK61359. Soraprazan is known to be a gastric H+/K+ ATPase inhibitor and act as an inhibitor of gastric acid secretion. Soraprazan is one of a preferable compound in the present inventio.

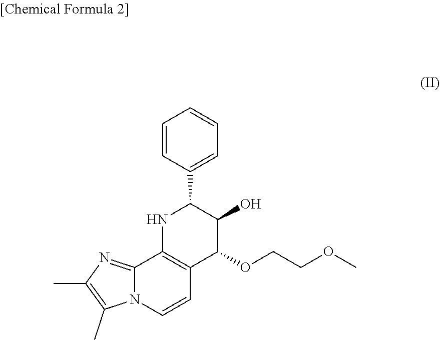

In one of the embodiments, the compound according to the present invention can be used for treatment and/or prevention of neuronal ceroid lipofuscinosis. Neuronal ceroid lipofuscinosis is a disease caused by systemic accumulation of lipofuscin in lysosomes of the patient's cells. The compound according to the present invention can reduce the amount of aberrantly accumulated lipofuscin in the cells, and thus can be used for the treatment of neuronal ceroid lipofuscinosis. Further, the compound according to the present invention can inhibit new accumulation of lipofuscin in the lysosome of the cell, and thus can also be used for the prevention of neuronal ceroid lipofuscinosis. In addition, the compound according to the present invention can be used for the treatment and/or prevention of not only neuronal ceroid lipofuscinosis but also any one of diseases caused by the accumulation of lipofuscin in patient's body, tissues, organs such as the brain, or cells.

Neuronal ceroid lipofuscinosis, also called Batten disease, is classified into multiple types according to genes that show abnormalities, including an infant type (Santavuori-Haltia disease), a late infant type (Jansky-Bielshowsky disease), a juvenile type (Spielmeyer-Vogt disease), an adult type (Kufus disease or Parry disease), and the like. However, when referred to as “neuronal ceroid lipofuscinosis”, it includes all types of diseases as long as which develops physical symptoms due to accumulation of lipofuscin in lysosomes in the cells that form the patient's body tissue due to a congenital genetic abnormality. At least 14 mutations of variants in different genes are known to result in the excessive accumulation of lipofuscin in somatic tissues and organs, or/and central nervous system of the body.

When referred to as “treatment”, it means ameliorating an existing medical condition, and when referred to as “prevention”, it means reducing a severity of a serious medical condition and/or slowing progression of a disease.

The compound according to the present invention can be formulated as a pharmaceutical composition containing a therapeutically effective amount thereof for use in the treatment and/or prevention of at least one type of neuronal ceroid lipofuscinosis. In one of the embodiments, the compound according to the present invention may be in a form of a pharmaceutically acceptable salt. The term “salt” refers to a compound in which a cation and an anion are ionically bonded, including a combination of a cation and an anion that are ionized in an aqueous solution state. The pharmaceutically acceptable salt of the compound according to the present invention is not particularly limited, but may be, for example, hydrochloride, hydrobromide, hydroiodide, nitrate, carbonate, sulfate, or phosphate, and further, it is, for example, acetate, propionate, succinate, glycolate, gluconate, lactate, malate, tartrate, citrate, ascorbate, glucuronate, maleate, fumarate, pyruvate, aspartate, glutamate, benzoate, anthranilate, 4-hydroxybenzoate, phenylacetate, mandelate, sulfonate, stearate, alginate, β-hydroxybutyrate, salicylate, galactarate, oxalate, or malonate. It may be a metal salt containing an alkali metal or an alkaline earth metal, and further, it may be a metal salt containing a transition metal such as calcium, magnesium, potassium, sodium or zinc.

When referred to as “therapeutically effective amount”, it means an amount of a compound that is effective in alleviating a condition of the patient's disease when the compound has been administered to a patient. A therapeutic effect and toxicity of such a compound can be determined by standard pharmaceutical procedures using cultured cell or laboratory animals. Herein the cells used for the standard pharmaceutical procedures may be cells genetically modified to accumulate lipofuscin or cells obtained from patients of neuronal ceroid lipofuscinosis. The cells can be genetically modified to accumulate lipofuscin by functionally disturbing at least one gene whose disruption causes neuronal ceroid lipofuscinosis or accumulation of lipofuscin. The laboratory animals used for the standard pharmaceutical procedures may be knock-out animals wherein at least one gene causing neuronal ceroid lipofuscinosis has been disrupted. A dose of the compound is preferably adjusted to result in ED50 with little or no toxicity. The dose may be appropriately selected depending on a dosage form used, age and patient conditions, an administration route, and the like.

In one of the embodiments, the pharmaceutical composition containing the compound or the salt thereof as an active ingredient, may further contain one or more pharmaceutically acceptable additives. An additive referred to a substance other than the active ingredient contained in pharmaceutical composition, and includes at least one of a flavoring agent, a foaming agent, a fragrance, an excipient, an isotonic agent, a surfactant, an emulsifier, a colorant, a buffer, zinc or its salt, a preservative, an antioxidant, a pH adjusting agent, a stabilizer, a suspension agent, or a solubilizing agent, or includes a combination thereof.

Examples of the flavoring agent include, but are not limited to, sucrose, sorbitol, citric acid, tartaric acid, malic acid and the like.

Examples of the foaming agent include, but are not limited to, sodium hydrogen carbonate and the like.

Examples of the fragrance include, but are not limited to, lemon, orange, cherry, raspberry, menthol and the like.

Examples of the excipient include, but are not limited to, monosaccharide such as glucose, mannose, xylose, and trehalose, disaccharide such as lactose hydrate, maltose, and sucrose, sugar alcohol such as mannitol, glycerol, glycerin, sorbitol, and xylitol, crystalline cellulose, corn starch, potato starch and the like.

Examples of the isotonic agent include, but are not limited to, sodium chloride, monosaccharide such as glucose, mannose, xylose, and trehalose, disaccharide such as lactose hydrate, maltose, and sucrose, sugar alcohol such as mannitol, glycerol, glycerin, sorbitol, and xylitol, and the like.

Examples of the surfactant include, but are not limited to, benzalkonium chloride, benzethonium chloride, polyoxyethylene (40) monostearate (polyoxyl 40 stearate), sorbitan sesquioleate, polysorbate 20, polysorbate 80, polyoxyethylene (160) polyoxypropylene (30) glycol, sodium lauryl sulfate, glyceryl monostearate (glycerin monostearate), polyoxyethylene lauryl ether (lauromacrogol), and the like.

Examples of the emulsifier include, but are not limited to, monoglyceride, diglyceride, glycerin fatty acid ester, organic acid monoglyceride, polyglycerol fatty acid ester, propylene glycol fatty acid ester, polyglycerol condensed ricinoleic acid ester, sorbitan fatty acid ester, sucrose fatty acid ester, lecithin, enzymatically decomposed lecithin, polysorbate 20, polysorbate 80, carrageenan, guar gum, gum arabic, and the like.

Examples of the colorant include, but are not limited to, iron sesquioxide, yellow iron sesquioxide, brown iron oxide, black iron oxide, titanium oxide, edible lake pigment, riboflavin, sodium riboflavin phosphate, and the like.

Examples of the buffer agent include, but are not limited to, citric acid, succinic acid, fumaric acid, tartaric acid, ascorbic acid, acetic acid, phosphoric acid, glutamic acid, glutamine, glycine, aspartic acid, alanine, arginine, histidine, magnesium oxide, zinc oxide, magnesium hydroxide, phosphoric acid, boric acid, or salts thereof, and the like.

Examples of the preservative include, but are not limited to, paraoxybenzoic acid ester (paraben), benzalkonium chloride, chlorobutanol, cresol, sodium benzoate, benzyl alcohol, sodium dehydroacetate, and the like.

Examples of the antioxidant include, but are not limited to, citric acid, sodium nitrite, ascorbic acid, sodium edetate, soy lecithin, natural vitamin E, sodium metabisulfite, dibutyl hydroxytoluene, and the like.

Examples of the pH adjusting agent include, but are not limited to, lactic acid, succinic acid, citric acid, phosphoric acid, carbonic acid, tartaric acid, fumaric acid, acetic acid, glutamic acid, glutamine, glycine, aspartic acid, alanine, or arginine, histidine, or salts thereof, sodium hydroxide, hydrochloride, and the like.

Examples of the stabilizer include, but are not limited to, sodium pyrosulfite, sodium edetate, erythorbic acid, magnesium oxide, dibutylhydroxytoluene, and the like.

Examples of the suspension agent include, but are not limited to, crystalline cellulose, carmellose sodium, xanthan gum, agar, gum arabic, tragacanth gum, sodium carboxymethyl cellulose, methyl cellulose, and the like.

Examples of the solubilizing agent include, but are not limited to, ethanol, propylene glycol, polyethylene glycol, sorbitan sesquioleate, sorbitan laurate, sorbitan palmitate, glyceryl oleate, glyceryl myristate, polyoxyethylene lauryl ether, polyoxyethylene nonylphenyl ether, or glycerin, and the like.

When administered to a patient with neuronal ceroid lipofuscinosis, the pharmacological composition of the present invention can reduce lipofuscin accumulated in the patient's body. As a result, a therapeutic effect of the compound on the patient can be achieved. For example, the amount of lipofuscin accumulated in the patient's body, tissues including the brain, or cells can be reduced to less than ½, ⅓, ¼, ⅕, or 1/10 when a therapeutically effective amount of the pharmacological composition of the present invention is administered to a patient with neuronal ceroid lipofuscinosis. The reduction of the amount of lipofuscin in the patient's body can be examined by obtaining patient's cells or a tissue before and after the administration of the compound and measuring the amount of lipofuscin in the cells or the tissue. The amount of lipofuscin can be measured, for example, by the method described in Examples in this specification. The administration route of the compound to the patient is not particularly limited, but may be appropriately selected, according to the patient condition, from subcutaneous, intravenous, intramuscular, topical, oral, parenteral, transdermal, intraperitoneal, intraorbital, implantation, inhalation, arachnoid, intravesical, or intranasal administration, and the like.

A dosage form of the pharmaceutical composition of the present invention is appropriately selected according to the route of administration. The dosage form is preferably tablets, capsules, granules, powders, oral solutions, syrups, oral jelly, oral tablets, oral sprays, oral semi-solid preparations, orally disintegrating tablets, effervescent tablets, emulsions, mouthwash, injections, or inhalants. When the pharmaceutical composition is orally administered to pediatric patients, considering the easiness of administration, the dosage form is more preferably a syrup, an emulsion, or an orally disintegrating tablet. When the pharmaceutical composition is manufactured in such a form as a syrup, it contains preferably the fragrance.

When the dosage form of the pharmaceutical composition of the present invention is syrups, in addition to the active ingredient, for example, sucrose, sorbitol, or the like as a flavoring agent can be added, polyethylene glycol, sorbitan laurate, glycerin, or the like can be added as a solubilizer, and, when necessary, a preservative such as paraoxybenzoic acid ester (paraben), sodium benzoate, or benzyl alcohol can be added. In the case of a suspension syrup, in addition to the above additives, agar, arabia gum, xanthan gum, or the like can be added as a suspension agent.

When the dosage form of the pharmaceutical composition of the present invention is orally disintegrating tablets, in addition to the active ingredient, for example, sodium hydrogen carbonate or the like as a foaming agent can be added, citric acid or the like as an antioxidant can be added, and mannitol or the like as an excipient can be added.

The pharmaceutical composition of the present invention can be used in combination with enzyme replacement therapy. The enzyme replacement therapy may be effective in treating certain forms of neuronal ceroid lipofuscinosis. Even when the enzyme replacement therapy has been selected or has been conducted for the treatment of neuronal ceroid lipofuscinosis, the pharmaceutical composition of the present invention can be administered to the patient concomitantly with the enzyme replacement therapy. Such an administration of the pharmaceutical composition may promote or assist a therapeutic effect of the enzyme replacement therapy on a patient, or may reduce the frequency of administration of enzyme preparations in the enzyme replacement therapy.

### EXAMPLES

In the following, the present invention is described in more detail with reference to examples, using soraprazan as an example of the compound according to the present invention. However, it is not intended that the present invention be limited to the examples.

### Evaluation Test Using Pathological Model Cells

### [Example 1-1] Preparation of Pathological Model cells

Using mammalian cells in which a causative gene of neuronal ceroid lipofuscinosis has been knocked out as pathological model cells, a test can be performed in which an effect of removing lipofuscin accumulated in the cells by soraprazan is evaluated. For example, using PPT1 knockout (KO) HEK293T cells or PPT1 KO CHO cells, which are pathological model cells of neuronal ceroid lipofuscinosis type 1 (CLN1), a test can be performed in which an effect of removing lipofuscin accumulated in the cells by soraprazan is evaluated. Acquisition of a PPT1 KO cell line can be achieved using a conventionally known method.

### [Example 1-2] Preparation of Reagents

Soraprazan is dissolved in dimethyl sulfoxide (DMSO) to a concentration of 10 mM. Subsequently, the solution is diluted with DMSO 10⁻¹ times each time in logarithmic steps to prepare DMSO solutions of soraprazan having concentrations of 10 mM-1 μM. As soraprazan, commercially available products can be used, such as soraprazan (HY-100414, MedChemExpress) can be used. As DMSO, commercially available products can be used, such as DMSO, Anhydrous (D12345, Thermo Fisher Scientific) can be used.

### [Example 1-3] Cell Culture

Knockout cells are suspended to a certain concentration, for example, 0.2×10⁶ cells/mL, 0.3×10⁶ cells/mL, or 0.4×10⁶ cells/mL using a liquid culture medium, and this cell suspension is mixed with each one of the soraprazan DMSO solutions prepared in Example 1-2 or 100% DMSO at a volume ratio of 99:1 to prepare 6 types of culture solutions having final soraprazan concentrations of 100 μM-10 nM, and 0 M. A certain amount, for example, 12 mL or 2 mL, of each of the prepared cell suspensions is seeded in a flask or a 6-well tissue culture plate, respectively. Each of cell suspensions is seeded in three flasks or three wells. The flasks or the plates are placed in an incubator and cultured in a wet environment at 37° C. and 5% CO₂ for a certain time period, for example, 4 days. When the cell culture is performed by shake culture, an Erlenmeyer flask is preferable choice, and the flask is placed in a shaker, and is shaken at a constant rotation speed of, for example, 80 rpm. Corning® 125 mL Polycarbonate Erlenmeyer Flask with Vent Cap (Corning) is an example of preferable flask for shake culture. When the cell culture is performed by static culture, for example, T75 EasYFlask, TC Surface, FiterCap (Thermo Fisher Scientific) can be preferably used. As the liquid culture medium, commercially available products can be used. For example, when HEK293T cells are used as knockout cell lines, DMEM/High Glucose medium containing 10% FBS (Thermo Fisher Scientific) can be preferably used. When CHO cells are used as knockout cell lines, CD OptiCHO™ medium (Thermo Fisher Scientific) can be preferably used. As the incubator, Thermo Scientific™ Forma™ Steri-Cult CO₂ Incubator model 3307 (Thermo Fisher Scientific) can be preferably used, and, as the shaker, MaxQ™ 2000 CO₂ Resistant Digital Shaker (Thermo Fisher Scientific) can be preferably used.

### [Example 1-4] Lipofuscin Fluorescence Emission measurement

Relative amounts of lipofuscin accumulated in cells can be measured by observing green autofluorescence emitted from the cells using a fluorescence microscope as described below. The cells after culturing with or without the presence of soraprazan are washed with such as an appropriate culture medium or buffer solution, consecutively are used for measurement. A microscopic image of the cells cultured with or without the presence of soraprazan is taken. Then, under a fluorescence microscope, for example, excitation light with wavelength of 485 nm is irradiated to the cells and strength of emitted light from the cells with wavelength of 521 nm is measured. As a fluorescence microscope, for example, EVOS FLoid Imaging System (Thermo Fisher Scientific) can be used, and in this case, wavelengths of excitation light and observation light can be respectively set to 482 nm and 532 nm. In addition, the wavelengths of the excitation light and the observation light can be changed appropriately depending on measuring equipment or a measurement condition, and a filter or the like can also be used as appropriate.

### [Example 1-5] Comparison of Measurement Results

Based on the microscopic images obtained in Examples 1-4, a fluorescence intensity of each of the microscopic images of the cells cultured in the presence of soraprazan are compared with that of the cells cultured in the absence of soraprazan (100% DMSO added). As the intensity of the emitted light has positive correlation with the amount of lipofuscin, it can be confirmed that the amount of lipofuscin accumulated in the cells decreases according to the concentration of soraprazan in the culture media. In this way, a test can be performed to evaluate the effect of soraprazan on removing lipofuscin accumulated in the cells.

### In Vitro Pharmacological Test Using CLN Patient-Derived Cell

### [Example 2-1] Expansion of CLN2 Patient-Derived Cells and Control Cells

Ceroid lipofuscinosis type 2 patient-derived fibroblasts (CLN2 cells, 7 donors: GM09668, GM09669, GM09404, GM10570, GM10672, GM16485, GM16486, Coriell Institute) and normal human dermal fibroblasts (NHDF cells, Cat.No.C-12300, PromoCell GmbH) were cultured in MEM (10370088, Gibco) containing 15% FBS (HyClone) and 1×GlutaMAX™ I (Gibco) (hereafter referred to as MEM). Each cell was diluted to 0.5×10⁵ cells/mL with MEM and 15 mL of each cell suspension was seeded in T75 flask (3123-075, IWAKI Inc.). Cells were cultured under 37° C., 5% CO₂ conditions.

### [Example 2-2] Preparation of Soraprazan-Containing Medium

Soraprazan (Symeres) was dissolved in Dimethyl sulfoxide (DMSO) to prepare soraprazan stock solution with concentrations of 1, 10, and 100 mM. Each soraprazan stock solution was diluted 1/100 concentration with MEM to prepare media with final concentrations of 0.1, 1, and 10 μM of soraprazan (0.0367, 0.367, and 3.67 μg/mL, respectively). In addition, DMSO was diluted to 1/100 concentration with MEM, which was used as a negative control (0 μM soraprazan). Hereinafter the prepared media herein were referred to as soraprazan-containing media collectively.

[Example 2-3] Seeding and culturing of cells with soraprazan-containing medium

Each of the CLN2 cells and the NHDF cells cultured in Example 2-1 was washed twice with Dulbecco's Phosphate-Buffered Saline (DPBS, Gibco) and detached from the flask using 1 mL of 0.25% Trypsin-EDTA (Gibco). After 10 mL of MEM was added, each cell was collected in a 15 mL tube and centrifuged at room temperature at 1000 rpm for 5 minutes, and the supernatant was removed. Then, each cell was suspended at cell density of 2×10⁵ cells/mL in one of soraprazan-containing media (0, 0.1, 1, and 10 μM). Then the cell suspension was dispensed in 96-well plates (655956, Gliner) with 0.4×10⁵ cells per well (200 μL per well). Cells were cultured at 37° C., in the presence of 5% CO₂ for 10 days. During the culture, the medium in each well was exchanged to new corresponding soraprazan-containing medium at 3, 6, and 9 days after seeding.

### [Example 2-4] Observation of Autofluorescent Material

Autofluorescent material in each cell was observed with Fluorescence Microscopy BZ-X700 (Keyence) at 10 days after cell seeding. The observation of autofluorescence was performed after changing the medium to FluoroBrite DMEM (A1896701, Gibco) under GFP detection conditions (BZX filter GFP, excitation wavelength:470/40 nm, absorption wavelength:525/50 nm, dichroic mirror: 495 nm).

### [Example 2-5] Results

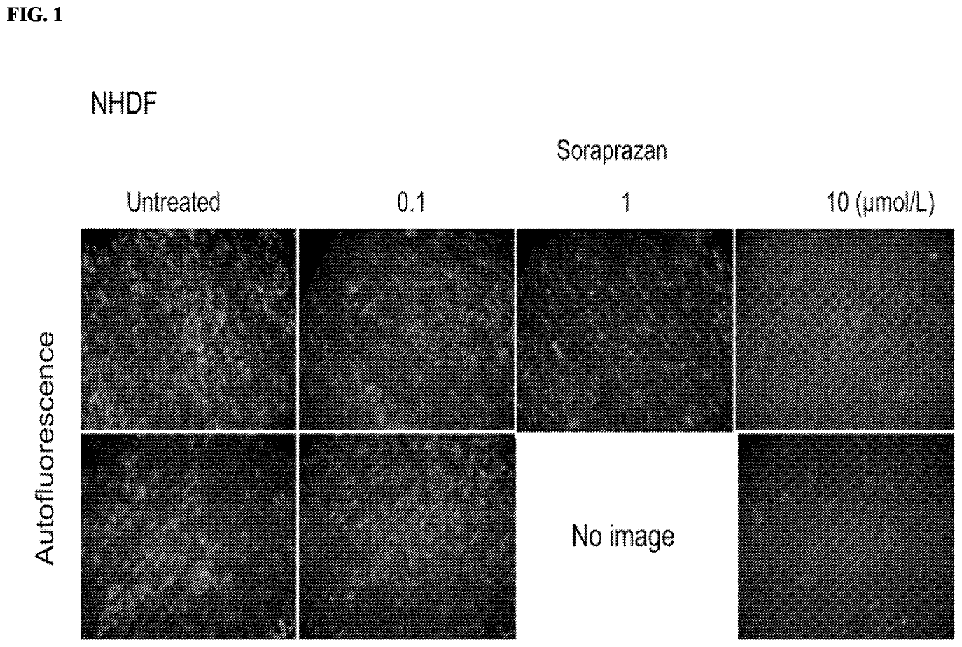

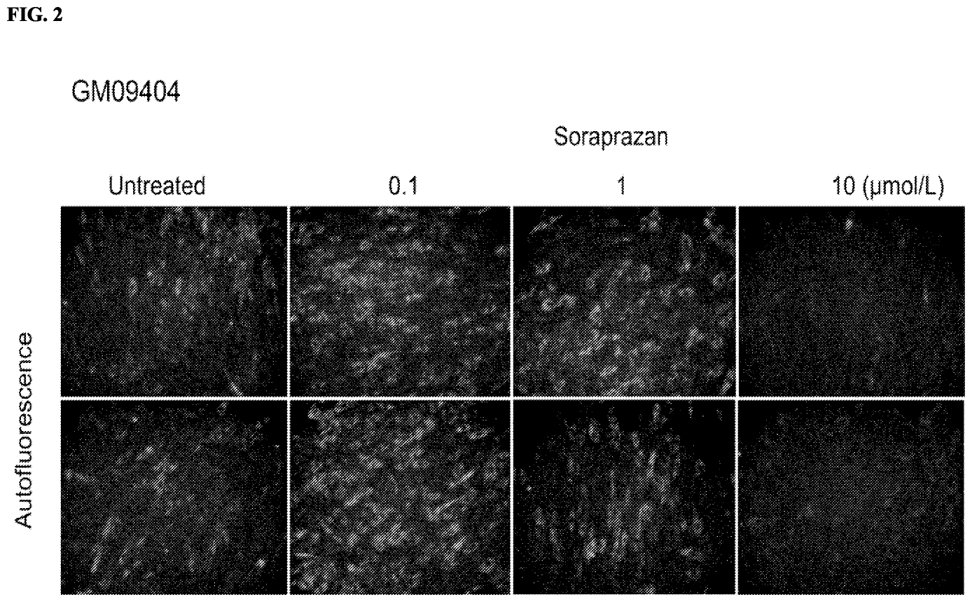

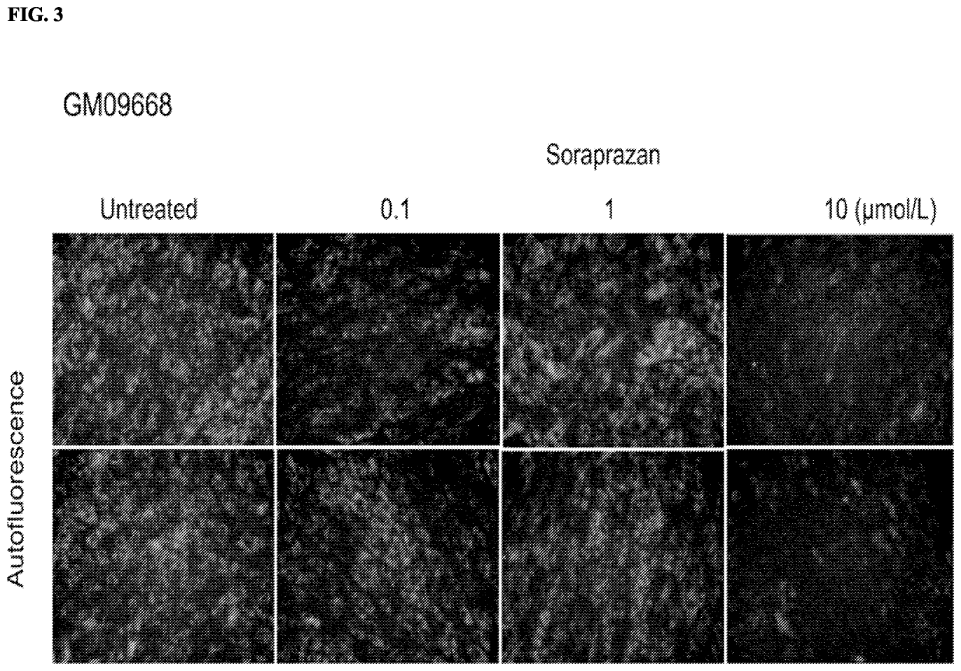

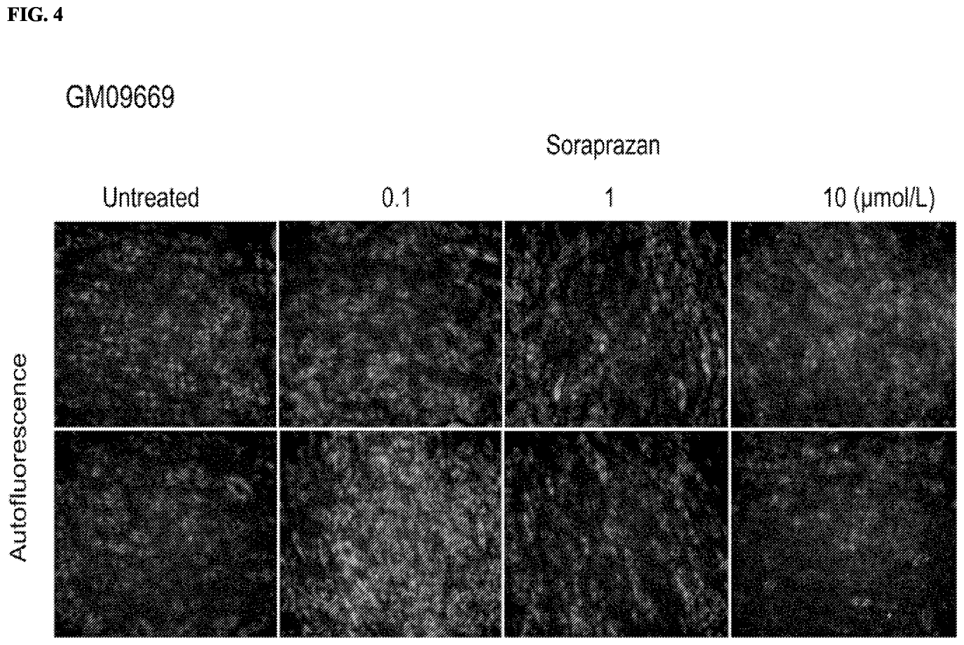

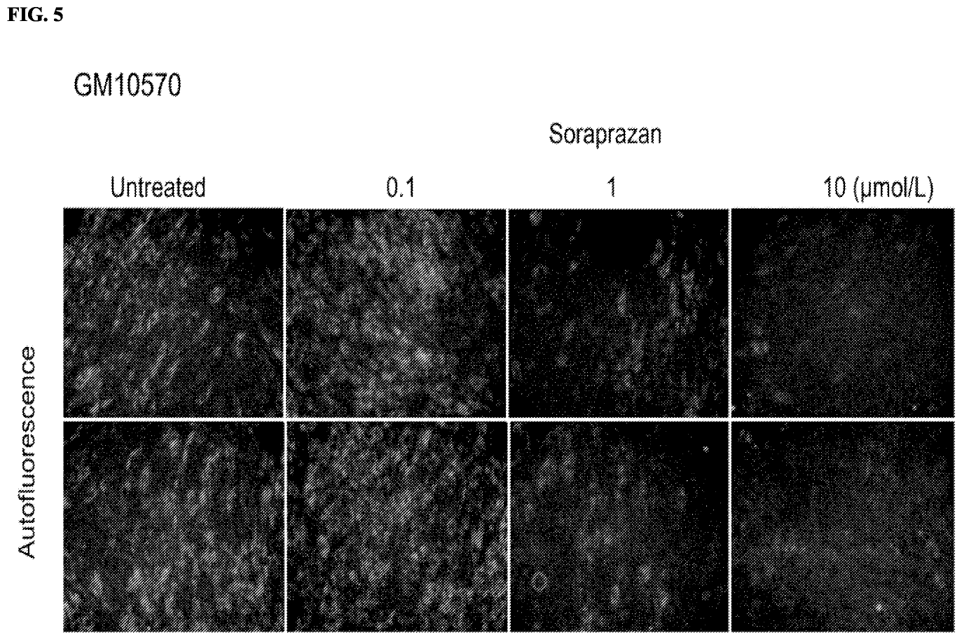

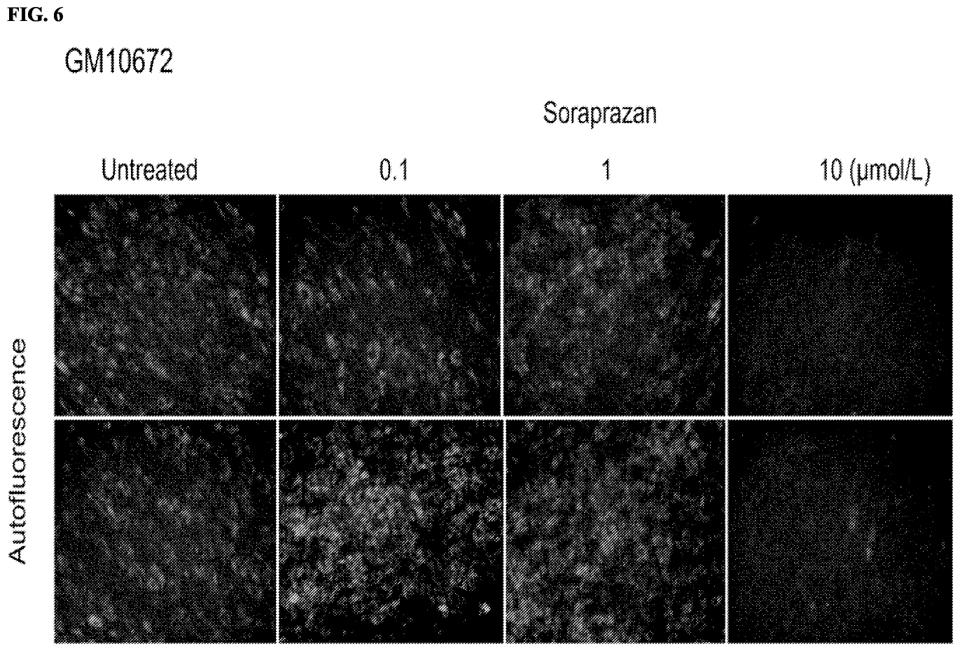

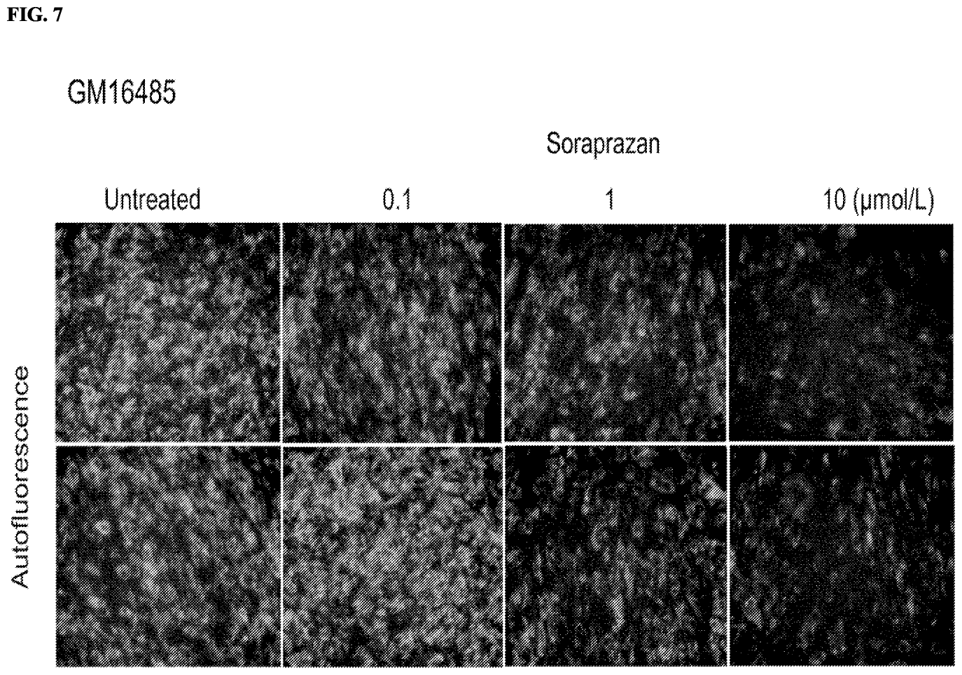

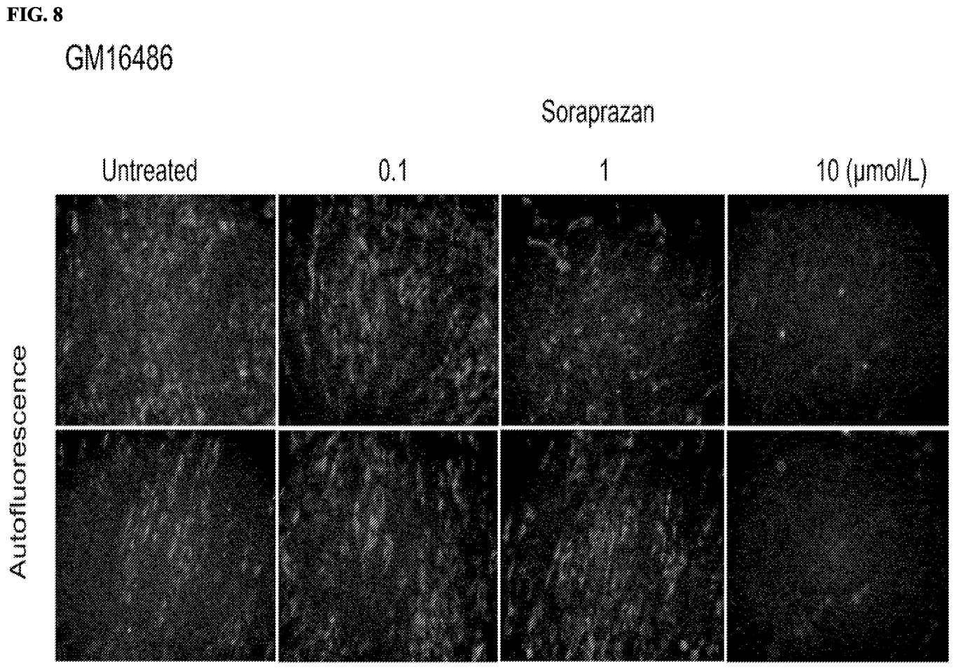

Results of autofluorescence observation obtained in Example 2-4 are shown in FIGS. 1-8. Except for NHDF cells, a negative control cell, the fluorescent intensity was markedly reduced in CLN2 cells cultured with soraprazan-containing medium, particularly at a concentration of 10 μM. This result indicates that the presence of soraprazan reduces the accumulation of lipofuscin in CLN2 cells.

### [Example 2-6] Quantification of Fluorescence Intensity

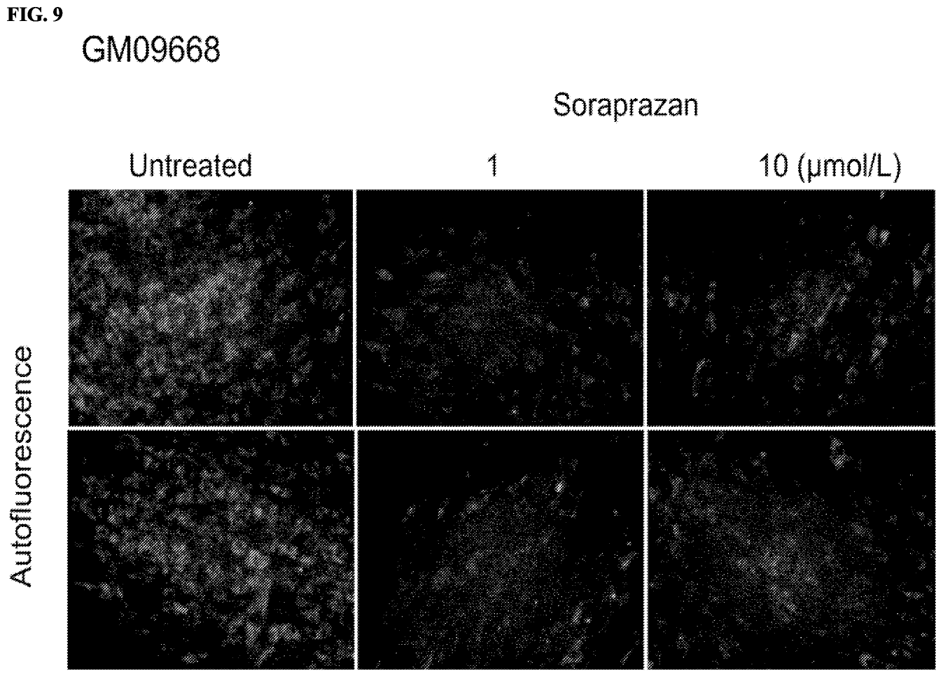

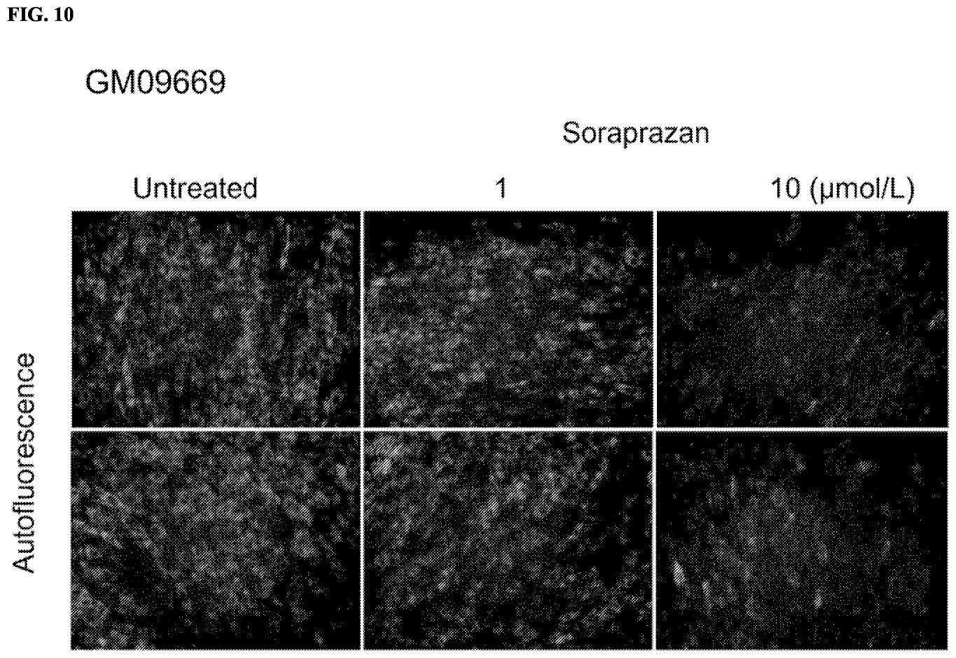

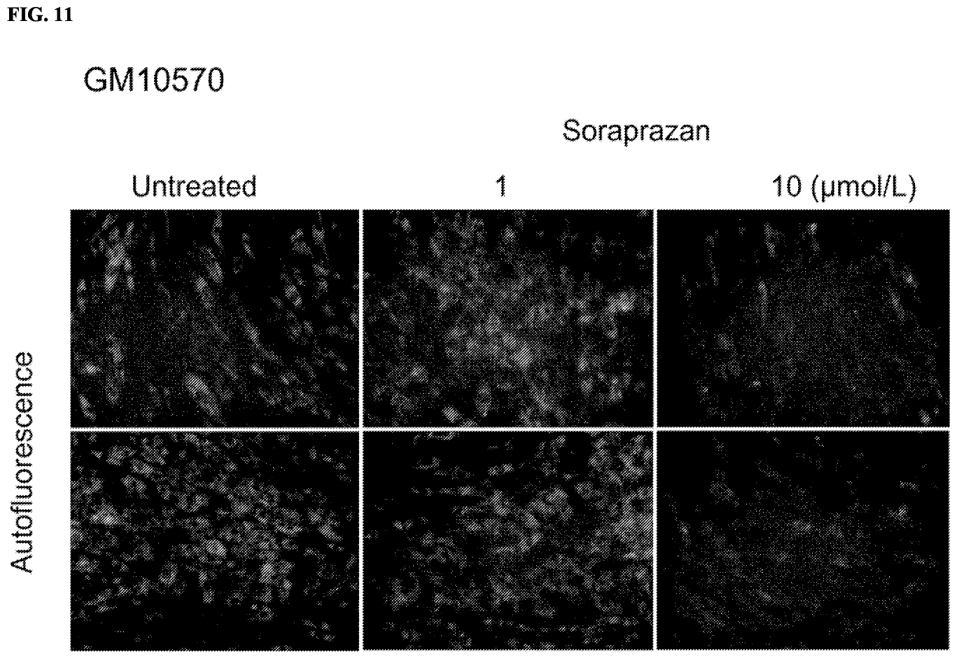

To quantify the fluorescence intensity of autofluorescence, the same tests as in Examples 2-1 to 2-4 were performed on 3 types of CLN2 cells (GM09668, GM09669, GM10570) using soraprazan-containing medium, which contained 0, 1, and 10 μM of soraprazan, respectively. The results of autofluorescence observations obtained are shown in FIGS. 9-11. It was confirmed again that the fluorescent intensity was reduced in cells cultured in soraprazan-containing medium particularly at a concentration of 10 μM. Then, the ratio of the area where fluorescent was detected in the image (fluorescent area) to the total area of the image occupied by the cells (% Fluorescent Area) was calculated using the image analysis software HALO® (v3.2.1851.354, Indica Labs). As the analysis algorithms, Indica Labs-Area Quantification FL v2 1.6 was used. Herein the area where fluorescent was detected, irrespective of the fluorescent intensity, was defined as fluorescent area, which corresponds the area where a certain amount of lipofuscin existed. The results are given in Table 1.

TABLE 1
Fluorescence intensity of autofluorescence

| | % Fluorescent Area | | |
|---|---|---|---|
| Soraprazan (μM) | GM09668 | GM09669 | GM10570 |
| 0 | 12.2 | 12.7 | 7.83 |
| 1 | 0.944 | 10.9 | 12.3 |
| 10 | 2.07 | 2.34 | 0.766 |

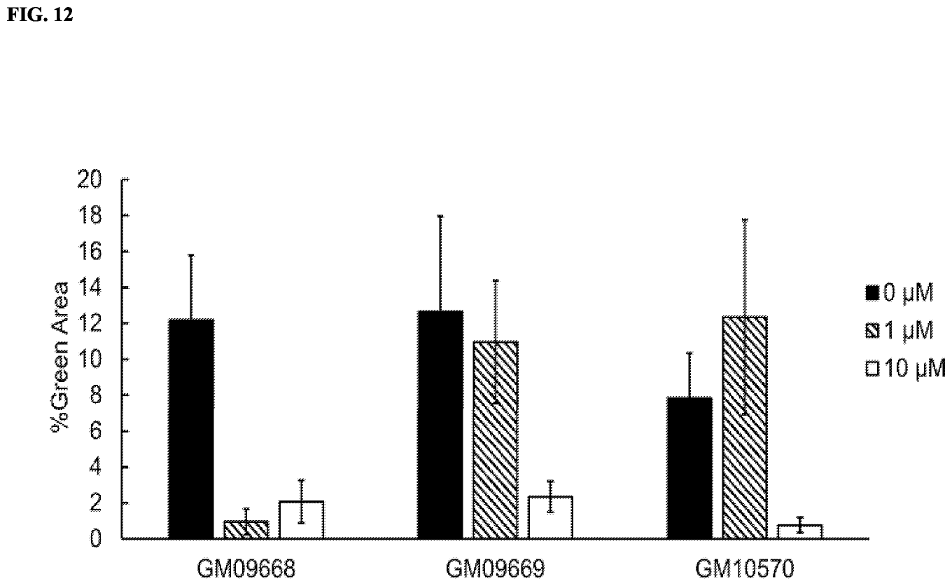

The results in Table 1 are depicted as a bar graph in FIG. 12. These results indicate that the amount of lipofuscin accumulated in the cells was reduced to almost ⅕ to 1/10 or less, when CLN2 cells were cultured in the medium containing 10 μM of soraprazan compared to those cultured in the absence of soraprazan. This suggests that lipofuscin accumulated in the cells of the patient should be reduced when therapeutically effective amount of soraprazan is administered to patients with neuronal ceroid lipofuscinosis or to patients with diseases in which lipofuscin accumulates in the body.

### Administration Test Using Pathological Model Animals

### [Example 3-1] Preparation of Experimental Animals

A test can be performed in which an effect of soraprazan on removing lipofuscin accumulated in animal tissues by soraprazan is evaluated using mammals in which a causative gene of neuronal ceroid lipofuscinosis has been knocked out as pathological model animals. For example, a test can be performed in which an effect of soraprazan on removing lipofuscin accumulated in mouse tissues by soraprazan is evaluated using TPP1 knockout (KO) mice, which are neuronal ceroid lipofuscinosis type 2 (CLN2) pathological model cells. Generation of the TPP1 KO mice can be performed using a conventionally known method.

### [Example 3-2] Administration of Soraprazan

Soraprazan is suspended in a 0.5% aqueous solution of methyl cellulose, and the resulting suspension is orally administered to a TPP1 KO mouse administration group at an appropriate dose, for example, 0.01 mg/kg to 10 mg/kg. Or, soraprazan is dissolved in physiological saline with or without additional solubilizers or formulation excipients, and the resulting solution is intravenously administered to a TPP1 KO mouse administration group at an appropriate dose, for example, 0.1 mg/kg to 10 mg/kg. The intravenous administration can be performed, for example, on the tail vein, the cephalic vein, or the saphenous vein. Further, a solution that does not contain soraprazan, such as physiological saline, is administered to a negative control group at the same dose. The administration can be repeated twice daily, daily, two or four times a week for time intervals between one week and 12 months.

### [Example 3-3] Measurement of Fluorescence Intensities of Tissues

After a certain time period, for example 0.25, 0.5, 1, 2, 4, 6, 8, or 24 hours after the last administration of a chronic treatment between one week and 12 months, all TPP1 KO mice are anesthetized with isoflurane and perfused with saline from the left ventricle for 4-5 minutes for blood removal. After that, tissues (brain, heart, lungs, liver, spleen, kidneys, quadriceps, thymus, thoracic vertebrae, femur) and peripheral blood, of which fluorescence intensities are to be measured, are collected, and are stored in physiological saline to prevent drying. Fluorescence intensity measurement of lipofuscin is performed using an appropriate fluorescence intensity measuring instrument. For example, a filter set for the in vivo emission and fluorescence imaging system IVIS LuminaIII (PerkinElmer) and the fluorescent dye VivoTag750 can be used to measure the fluorescence intensity according to operating instructions.

### [Example 3-4] Comparison of Measurement Results

Regarding fluorescence intensity images obtained in Example 2-3, using images of the tissues of the negative control group as references, fluorescence intensities of the tissues of the soraprazan-administered group are compared, and thereby, it can be confirmed that soraprazan reduces the amount of lipofuscin accumulated in each of the tissues. In this way, a dose-dependent effect of removing lipofuscin accumulated in animal tissues by soraprazan can be evaluated. Then, based on a result of the evaluation, a suitable dosing plan can be made when administering soraprazan as a therapeutic or prophylactic agent for neuronal ceroid lipofuscinosis.

### Industrial Applicability

According to the present invention, a novel therapeutic compound for neuronal ceroid lipofuscinosis can be provided.

1. A compound for treatment and/or prevention of neuronal ceroid lipofuscinosis represented by the following general formula [I], wherein
in the general formula [I], R1 is a methyl group or a hydroxymethyl group, R2 is a methyl group or a hydroxymethyl group, R3 is one of a hydroxy group, a methoxy group, an ethoxy group, an isopropoxy group, a methoxyethoxy group, a methoxypropoxy group, or an ethoxypolopoxy group, and R4 is one of a hydroxy group, a methoxy group, an ethoxy group, an isopropoxy group, a methoxyethoxy group, a methoxypropoxy group, or an ethoxypolopoxy group.

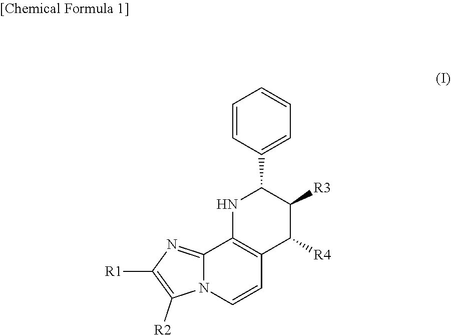

2. The compound according to claim 1, wherein, in the general formula [I], R1 is a methyl group.

3. The compound according to claim 1, wherein, in the general formula [I], R1 is a methyl group, and R2 is a methyl group.

4. The compound according to claim 1, wherein, in the general formula [I], R3 is a hydroxy group.

5. The compound according to claim 1, wherein, in the general formula [I], R3 is a hydroxy group, and R4 is a methoxyethoxy group.

6. The compound according to claim 1, wherein, in the general formula [I], R1 is a methyl group, R2 is a methyl group, R3 is a hydroxy group, and R4 is a methoxyethoxy group.

7. A pharmaceutical composition for treatment and/or prevention of neuronal ceroid lipofuscinosis, comprising a therapeutically effective amount of the compound according to claim 1 or a pharmaceutically acceptable salt thereof.

8. The pharmaceutical composition according to claim 7 characterized in that, when administered to a patient with neuronal ceroid lipofuscinosis, lipofuscin accumulated in the patient's body is reduced.

9. The pharmaceutical composition according to claim 8, wherein the administration is subcutaneous, intravenous, intramuscular, parenteral, local, oral, transdermal, intraperitoneal, intraorbital, implantation, inhalation, intraarachnoid, intravesical, or intranasal administration.

10. The pharmaceutical composition according to claim 7, being in a dosage form selected from a group consisting of tablets, capsules, granules, powders, oral solutions, syrups, oral jelly, oral tablets, oral sprays, oral semi-solid preparations, orally disintegrating tablets, parenteral emulsions, parenteral suspensions, parenteral solutions, effervescent tablets, mouthwash, injections, and inhalants.

11. The pharmaceutical composition according to claim 7, further comprising at least one pharmaceutically acceptable additive.

12. The pharmaceutical composition according to claim 11, wherein the at least one additive is selected from a group consisting of a flavoring agent, a foaming agent, a fragrance, an excipient, an isotonic agent, a surfactant, an emulsifier, a colorant, a buffer, zinc or its salt, a preservative, an antioxidant, a pH adjusting agent, a stabilizer, and a suspension agent or a solubilizing agent.

13. The pharmacological composition according to claim 7, being used together with enzyme replacement therapy for a patient with neuronal ceroid lipofuscinosis.
